(19) United States
(12) Patent Application Publication
(10) Pub. No.: US 2014/0230089 A1
(43) Pub. Date: Aug. 14, 2014

(54) METHOD FOR IMPROVING THE RESISTANCE OF PLANTS TO VIRUSES

(71) Applicants: Christian Chevalier, Villefranche De Lonchat (FR); Veronique Decroocq, Castres (FR); Frederic Delmas, Gradignan (FR); Michel Hernould, ST Jean D'Illac (FR); Julie Leblond, Bordeaux (FR); Adrien Sicard, Postdam-Golm (FR)

(72) Inventors: Christian Chevalier, Villefranche De Lonchat (FR); Veronique Decroocq, Castres (FR); Frederic Delmas, Gradignan (FR); Michel Hernould, ST Jean D'Illac (FR); Julie Leblond, Bordeaux (FR); Adrien Sicard, Postdam-Golm (FR)

(73) Assignee: INSTITUT NATIONAL DE LA RECHERCHE AGRONOMIQUE, Paris Cedex 07 (FR)

(21) Appl. No.: 14/131,529

(22) PCT Filed: Jul. 10, 2012
(86) PCT No.: PCT/EP2012/063513

§ 371 Date: Feb. 25, 2014

(30) Foreign Application Priority Data
Jul. 11, 2011 (FR) 1156258

(51) Int. Cl. C12N 15/82

(52) U.S. Cl. CPC C12N 15/8283
USPC 800/279

(57) ABSTRACT

A method for improving the resistance of a plant to viruses, includes modifying the genome of the plant so as to at least partially inhibit the activity of a CSN5 protein of the plant. The modification of the genome of the plant can involve mutating a gene of the plant that encodes the CSN5 protein, or can be selected so as to cause the overexpression of a mini zinc finger (MIF) protein.

The present invention relates to the field of combating infections of plants with RNA viruses. More particularly, it relates to a method for improving the resistance of plants to RNA viruses.

Plant diseases induced by RNA viruses, and notably by potyviruses, lead to large economic losses for many crops, by reducing yields and/or quality.

To date, there are few strategies for combating these infections. In particular, at present there is no chemical or biological treatment for preventing, treating or even limiting them. The most effective method currently employed is uprooting and destruction of infected plants. This method requires deploying a great deal of manpower, and it is unsatisfactory from an economic standpoint.

Strategies have been proposed in the prior art for creating transgenic plants having increased resistance to viral infections. However, these strategies are specific to certain viruses and/or to certain cultivated plants, and they cannot be generalized for all viruses and for all plants. Moreover, the viruses may be able to evade the resistance that has been conferred, making these combating means ineffective.

The aim of the present invention is to propose a solution to the problem of infection of plants by RNA viruses, which is applicable to all eukaryotic vegetable organisms and which is not specific to a particular type of virus.

For this purpose, the present inventors have advantageously profited from their recent discovery that the CSN5 protein, subunit 5 of the COP9-signalosome complex of plants, called the CSN complex, plays an important role in the mechanisms of regulation of the interactions between plants and pathogenic viruses, more particularly with regard to RNA viruses.

The COPS-signalosome complex is a multi-protein complex evolutionarily conserved in eukaryotes, comprising eight subunits, and having a central role in the ubiquitin-proteasome pathway. In plants, this complex is described as having deneddylation activity as its principal biological function. This activity is mediated by subunit 5 of the complex (CSN5) (Schwechheimer et al., 2010). This CSN5 protein is highly conserved in all the higher eukaryotes.

More particularly, the present inventors discovered that a decrease in activity of the CSN5 protein in plants led to greatly increased resistance of these plants to RNA viruses.

Thus, according to the present invention, a method is proposed for improving a plant's resistance to RNA viruses, which comprises a step of modifying the plant genome so as to inhibit, at least partially, the activity of a CSN5 protein of the plant, in particular its protease activity, and even more particularly so as to reduce its isopeptidase activity.

Preferably, this method further comprises a step of selecting, from the mutant plants thus obtained, mutant plants that are viable and fertile, and have greater resistance to viruses than the wild-type plant.

This step of selecting the mutant plants that have increased resistance to viruses is carried out according to the conventional criteria of resistance to viruses, in particular by inoculation with the virus, if necessary fused with a fluorescent marker, and detection of the virus. Said detection can for example be performed by the conventional techniques of enzyme-linked immunosorbent assay (ELISA), by reverse transcription followed by polymerase chain reaction (RT-PCR), or by observation with the microscope of the fluorescence produced by a viral inoculum expressing a fluorescent marker, such as the Green Fluorescent Protein (GFP).

The method according to the invention advantageously makes it possible, for a given plant, to obtain mutant plants in which the activity of the CSN5 protein is decreased or suppressed and whose resistance to viruses is improved relative to the wild-type plant.

This method is advantageously applicable to various species of plants, notably, but not limited to, the crucifers (or Brassicas) and the tomato.

In general, a person skilled in the art will easily be able to identify, depending on the plant in question, the corresponding particular CSN5 protein, and in particular the gene or genes encoding this protein, from information currently available in databases of DNA sequences or of protein sequences. More particularly, he will be able to identify in the databases, for a plant in question, by searches based on sequence analogy (BLAST), the homolog or homologs to the CSN5 proteins of the tomato or of Arabidopsis existing in these databases.

In general, whatever the plant in question, the CSN5 protein will have at least 50% identity, and even at least 80% identity, with peptide sequence SEQ ID NO: 13 or peptide sequence SEQ ID NO: 14. These sequences correspond respectively to the proteins called CSN5-1 and CSN5-2 of the species Solanum lycopersicum (tomato).

Otherwise, the CSN5 protein will have at least 50% identity, and even at least 80% identity, with peptide sequence SEQ ID NO: 15 or peptide sequence SEQ ID NO: 16. These sequences correspond respectively to the proteins called CSN5A and CSN5B of the species Arabidopsis thaliana.

The two CSN5 proteins of Arabidopsis (CSN5A and CSN5B) have high percentage identities, above 80%, with the two CSN5 proteins of tomato, as illustrated in FIG. 2, which shows alignment of the primary sequences of the CSN5 proteins of Arabidopsis thaliana and of tomato (Solanum lycopersicum, also called Lycopersicon esculenturn).

The method according to the invention makes it possible to endow the plants to which it is applied with resistance to various types of viruses, more particularly to RNA viruses, and in particular to the potyviruses, for example the Plum Pox Virus (PPV), the Turnip Mosaic Virus (TuMV) or the Lettuce Mosaic Virus (LMV).

The plant genome is preferably modified so as to partially inhibit the activity of the CSN5 protein, more particularly its isopeptidase activity.

In preferred embodiments of the invention, the modification of the plant genome is a mutation, and can be a point or insertion mutation, of a gene of the plant encoding the CSN5 protein, said modification preferably being selected for inducing either a decrease in accumulation of the CSN5 protein, or synthesis of a truncated CSN5 protein with reduced isopeptidase activity. Preferably, such a mutation will be selected for directly affecting the active site of the isopeptidase activity of the CSN5 protein, or for affecting a site involved in interaction of the CSN5 protein with other proteins involved in its isopeptidase activity, for example with other constituents of the signalosome complex, with a target protein of this isopeptidase activity, or with a protein with which the CSN5 protein interacts and then relocalizes in the cell.

In general, the techniques of manipulation of plants employed in the context of the present invention are conventional per se, and form part of the general knowledge of a person skilled in the art.

In particular, mutation of a gene encoding the CSN5 protein can be effected by any conventional technique per se. For example, mutants can be obtained by inserting transfer DNA (T-DNA) in the gene, and selecting, from the defective mutants thus obtained, for which a decrease in expression of the CSN5 gene is observed, or else giving rise to expression of a nonfunctional truncated CSN5 protein, mutants having a capacity for resistance to viruses greater than that of the wild-type plant. Advantageously, the method preferably further comprises an additional step of selecting, from the mutant plants identified with improved capacity for resistance to viruses, mutant plants which, as well as being viable and fertile, display capacity for growth allowing them to be cultivated in economically profitable conditions.

These mutants can otherwise be obtained by random mutagenesis of the plant DNA and selected after identification of the mutants bearing a mutation on the gene encoding the CSN5 protein, by application of the conventional technique known as TILLING: “Targeting Induced Local Lesions in Genomes” (MacCallum et al., 2002; Henikoff et al., 2004). TILLING is a reverse genetics technique utilizing the capacity of an endonuclease for detecting the mispairings in a DNA double strand and for performing cleavage at unpaired bases, for detecting mutation points generated by treating a plant with a mutagenic chemical. This technique is particularly suitable for applying high-throughput screening methods for selecting plants having a mutation induced by chemical mutagenesis in a target gene.

Thus, in preferred embodiments of the invention, the method for improving a plant's resistance to RNA viruses comprises the steps of generating a collection of mutant plants by chemical mutagenesis, and of selecting, from the collection of mutant plants thus generated, viable plants possessing a mutation on said gene of the plant encoding said CSN5 protein and whose capacity for resistance to RNA viruses is greater than that of the wild-type plant.

The expression of the CSN5 gene can be verified by any technique known by a person skilled in the art, for example by reverse transcription followed by quantitative polymerase chain reaction (quantitative RT-PCR).

According to the invention, the mutants defective for the gene encoding the CSN5 protein are preferably, but not exhaustively, homozygotic.

In preferred embodiments of the invention, the mutation of the gene encoding the CSN5 protein is introduced into an exon of the gene. The invention does not however exclude the mutation being located in an intron of the gene, if this mutation affects the activity of the CSN5 protein encoded by this gene, in the sense recommended by the invention.

Preferably, when the plant is the tomato, inhibition of the activity of the CSN5 protein can just as well be obtained by mutation of one and/or other of the two genes encoding a CSN5 protein and identified under the respective GenBank accession numbers AK328186.1 (Solanum lycopersicum, GI: 225315036) and AF175964.1 (Solanum lycopersicum, GI: 12002864).

In the species Arabidopsis thaliana, inhibition of the activity of the CSN5 protein is preferably obtained by a mutation of the CSN5A gene of the plant. This gene, also called AJH1, is known per se and is identified by the GenBank accession number AT1 G22920.

Otherwise, this inhibition can be obtained by a suitable mutation of the CSN5B gene, also known by the name AJH2, and identified by the GenBank accession number AT1G71230.

When the plant is the peach tree (Prunus persica), the method according to the invention for improving the resistance of the plant to RNA viruses comprises modification of the plant genome by mutation of the gene encoding the CSN5 protein of peptide sequence SEQ ID NO: 19. This protein has for example 82% identity with peptide sequence SEQ ID NO: 15 and 83% identity with peptide sequence SEQ ID NO: 16, corresponding respectively to the CSN5A and CSN5B proteins of the species Arabidopsis thaliana.

In other preferred embodiments of the invention, modification of the plant genome is selected for leading to overexpression of a member of the Mini Zinc Finger protein family.

The Mini Zinc Finger proteins (MIF) are small proteins of about 100 amino acids, characterized by a motif of the type CX₃HX₁₁CX₁₂₋₂₆CX₂CXCHX₃H.

A family of MIF proteins (called MIF1, MIF2, MIF3) has notably been characterized in Arabidopsis (Hu and Ma, 2006). The MIF1 protein has notably been shown to be involved in multiple hormonal regulations during development of the plant, acting as an inhibitor of growth of the plant.

In the tomato, the gene IMA has been identified, for “Inhibitor of Meristem Activity” (GenBank accession No.: AM261628.1, GI: 118621154, Solanum lycopersicum), which encodes a Mini Zinc Finger protein having 62% identity with the MIF2 protein of Arabidopsis (Sicard et al., 2008), as illustrated in FIG. 3. This protein has notably been shown to control the development of the floral meristem.

Quite surprisingly, it was discovered by the present inventors that the MIF proteins, and notably the MIF2 protein in Arabidopsis (encoded by the MIF2 gene identified in GenBank by the accession No. NM_{—}202644.1, GI: 42572554, Arabidopsis thaliana), interacts with the CSN5 protein, and that overexpression of a MIF protein induces within the plant a decrease in activity of the latter, leading to increased resistance of the plant to viral infections. Thus, the plants overexpressing the MIF protein, for which it was observed by the present inventors that they partially phenocopy the mutants that are defective for a CSN5 gene, have a higher level of resistance to RNA viruses than the wild-type plants.

Once again, a person skilled in the art will easily be able to identify, depending on the plant in question, the particular corresponding protein or proteins that are members of the family of the MIF proteins, and if applicable the gene or genes encoding this protein or these proteins, from information currently available in the databases of DNA sequences or of protein sequences.

In general, for each plant in question, the protein that is a member of the Mini Zinc Finger protein family has at least 60% identity with peptide sequence SEQ ID NO: 17 (corresponding to the MIF2 protein of Arabidopsis thaliana) or peptide sequence SEQ ID NO: 18 (corresponding to the protein encoded by the IMA gene of the tomato).

In preferred embodiments of the invention, when the plant belongs to the species Arabidopsis thaliana, inhibition of the activity of the CSN5 protein is achieved by overexpression of the MIF2 protein of sequence SEQ ID NO: 17. When the plant is the tomato, modification of the plant genome is selected for leading to overexpression of the protein encoded by the IMA gene, of sequence SEQ ID NO: 18, a functional homolog of the MIF2 protein of Arabidopsis.

This modification of the plant genome can be effected by any conventional method per se, notably by introducing an expression vector in cells of the plant for overexpressing the Mini Zinc Finger protein (Sicard et al., 2008).

### BRIEF DESCRIPTION OF THE DRAWINGS

FIGS. 1a and 1b are diagrams illustrating, for the mutants of Arabidopsis thaliana csn5a-1, csn5a-2, MIF2OE3, MIF2OE4, MIF2OE6, MIF2OE7 and for the wild-type line, expression of a gene CSN5 measured by quantitative RT-PCR, CSN5A for FIG. 1a and CSN5B for FIG. 1b.

FIG. 2 shows an alignment of the primary sequences of the CSN5 proteins of Arabidopsis thaliana (proteins CSN5A and CSN5B) and of tomato (Solanum lycopersicum CSN5-1 and CSN5-2), the active site that is the location for the isopeptidase activity of the protein being delimited by a box.

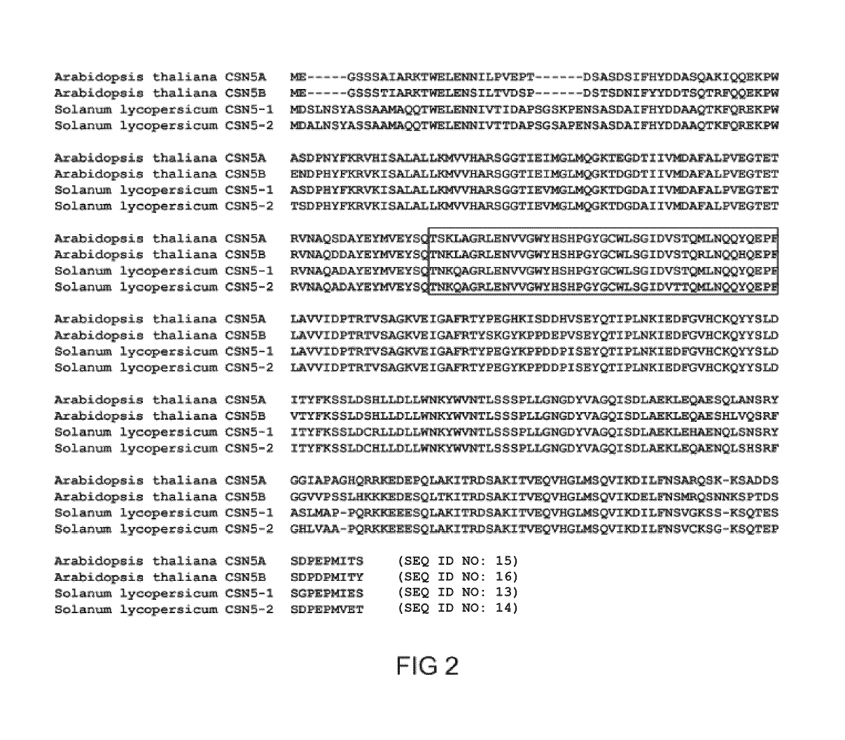

FIG. 3 shows an alignment of the primary sequences of the Mini Zinc Finger 2 proteins of Arabidopsis thaliana (MIF2) and of tomato (Solanum lycopersicum MIF2/IMA), the sequence corresponding to the zinc finger being delimited by a box.

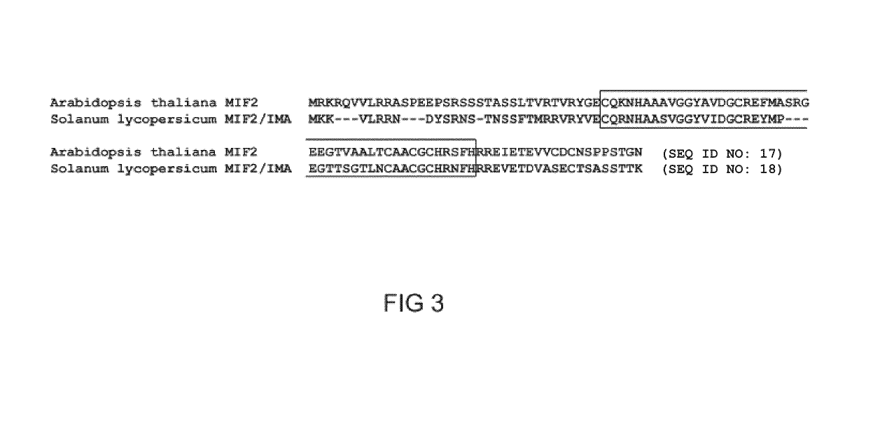

The invention will now be described more precisely in the context of the following examples, which do not in any way limit it.

Experiments

### A/PROTOCOLS OF ASSAYS FOR RESISTANCE TO THE POTYVIRUSES PLUM POX VIRUS (PPV), TURNIP MOSAIC VIRUS (TUMV) AND LETTUCE MOSAIC VIRUS (LMV) IN ARABIDOPSIS THALIANA

1/Viruses and Viral Strains Used

The isolates Rankovic (or PPV-R, belonging to the strain PPV-D also called Dideron) and PPV-NAT (Not Aphid transmissible, of the strain PPV-D), described in the works of Decroocq et al. (2006, 2009), are used.

The isolate PPV-R exists in the form of an infectious clone called pICPPV. This infectious clone was modified in order to insert a gene encoding the green fluorescent protein (GFP), to give the infectious clone pICPPVnk-GFP (Fernandez-Fernandez et al., 2001).

The viral construct [promoter CaMV35S-PPVnk-GFP-terminator] of pICPPVnk-GFP was transferred into a binary plasmid of the type pBIN19, giving rise to an infectious clone, agro-inoculable (i.e. inoculable in plants by transformation by Agrobacterium tumefaciens), called pBINPPVnk-GFP (cf. Decroocq et al., 2009).

The isolates TuMV-UK1 and CDN1 of the Turnip Mosaic Virus were also used (Jenner et al., 2000; Lehmann et al., 1997).

Finally, the isolate LMV-AF199 of the Lettuce Mosaic Virus was used in this study (Krause-Sakate et al., 2002).

2/Techniques for Inoculation of Arabidopsis

In parallel with the mutants tested, for each experiment it was used the Columbia accession as positive control, and as negative control, resistant to infection by most of the potyviruses, including PPV, the plant E6 (‘loss of function’ mutant obtained by insertion of T-DNA into the gene encoding elFiso4E) (cf. Decroocq et al., 2006; Duprat et al., 2002):

The resistance assays were carried out by mechanical inoculation and agro-inoculation for PPV, and by mechanical inoculation only, for TuMV and LMV.

2-1/Mechanical Inoculation

Young seedlings of Arabidopsis aged from 5 to 6 weeks post-sowing are inoculated mechanically by lightly rubbing the leaves with an inoculum consisting of a ground product of leaves of Nicotiana benthamiana previously infected with an isolate of PPV (natural isolates after mechanical propagation, or infectious clones, after biolistics with an infectious cDNA). Seedlings of Nicotiana benthanamia also serve as reservoirs for the AF199 isolate of the LMV. In the case of the TuMV, the isolates used (UK1 and CDN1) are propagated beforehand on turnip.

The leaves are ground in a mortar with 3 volumes of an inoculation buffer consisting of Na₂HPO₄ at 25 mM and of sodium diethyldithiocarbamate (DIECA) at 0.2% (w/v), with addition of an abrasive, carborundum. This abrasive damages the leaf, allowing the solution to penetrate more easily. The leaves are then rinsed with water to remove the excess and prevent burns. A second inoculation is carried out two days later. In order to study the systemic propagation of the virus, the young stems are cut just before inoculation.

2-2/Inoculation by Agro-Infiltration

The cDNA of the viral genome PPV-R cloned in the binary plasmid pBIN19 gave rise to the infectious clone pBINPPVnk-GFP. It is resistant to kanamycin and has a replication origin (ORI) compatible with Agrobacterium tumefaciens (Jimenez et al., 2006). pBINPPVnk-GFP is maintained in glycerol stock at a temperature of −80° C. in a strain of Agrobacterium C58C1, endowing it with resistance to tetracycline.

In order to start a new bacterial culture for inoculation of plants with the agro-inoculable infectious clone, the bacterial strain C58C1:pBINPPVnk-GFP is taken from the glycerol stock and spread on solid LB medium (Luria-Bertani medium, GIBCO-BRL: 2% Bactotrypone, 0.5% of yeast extract, 10 mM NaCl, 2.5 mM KCl), with addition of the antibiotics tetracycline 12.5 μg/ml and kanamycin 25 μg/ml. The bacterial strain is incubated for 48 h at 28° C. Two days later, a colony is taken and is incubated for 48 h at 28° C. in 5 ml of liquid LB medium, in the presence of the two antibiotics, so as to form a preculture.

Finally, the 5 ml of this preculture is transferred into 45 ml of liquid LB medium, with addition of 10 mM of 2-(N-morpholino)-ethane sulfonic acid (MES), 20 μM of acetosyringone, 25 μg/ml of kanamycin and 12.5 μg/ml of tetracycline. The bacterial culture is placed again at 28° C. for 24 h in a conical flask.

After 24 h, the 50 ml of culture is transferred to Falcon® tubes and centrifuged at 3900 rpm for 15 minutes. The pellet is rinsed with distilled water, resuspended and centrifuged again. This operation is repeated twice. After the two washings, the pellet is resuspended in an agro-infiltration solution comprising 10 mM of MES at pH 6.3, 10 mM of MgCl₂ and 150 μM of acetosyringone. Finally, the optical density of the solution at 600 nm (OD_{600 nm}) is adjusted to 0.6.

After incubation of the solution for 3 h at room temperature, the plants are inoculated by scarification using a toothpick previously impregnated with the bacterial solution.

3/Detection of the Viruses

PPV is detected by observations of the fluorescence produced by the virus, by enzyme-linked immunosorbent assay (ELISA) and reverse transcription and PCR (RT-PCR) as described below.

TuMV is detected by symptomatology and RT-PCR.

LMV is detected by ELISA assays.

3-1/Detection of the Virus by Enzyme-Linked Immunosorbent Assay (ELISA)

100 μl of a solution of IgG antibodies diluted to 1/1000 in carbonate buffer 1× (15 mM Na₂CO₃, 30 mM NaHCO₃) is adsorbed on ELISA plates, which are incubated for 3 h at 37° C. The plates are then rinsed 3 times for 3 minutes with PBS-Tween® buffer (136.9 mM NaCl, 1.47 mM KH₂PO₄, 2.68 mM KCl, 8.1 mM Na₂HPO₄, 0.05% (v/v) Tween®20).

The vegetable samples are ground in PBS-Tween®-PVP buffer 1× (PBS-Tween® buffer with addition of 21% (w/v) of polyvinylpyrrolydonedine (PVP) 25K), then 100 μl of this ground product is deposited in the wells of the plates. The latter are then incubated overnight at 4° C. The plates are rinsed again 3 times for 3 minutes with PBS-Tween® buffer. A conjugate is then diluted in PBS-Tween®-PVP-Ovalbumin buffer (0.2% (w/v) of ovalbumin), and 100 μl is deposited in each well. The plates are incubated for 2 h at 37° C. After 3 rinsings of 3 minutes, 100 μl of diethanolamine substrate buffer (9.7% (v/v) of diethanolamine, pH 9.8) with addition of one capsule of paranitrophenyl phosphate (pNPP, SIGMA, 1 mg/ml), is deposited on the plates. The optical density at a wavelength of 405 nm is measured with a SAFAS MP96 microplate reader.

3-2/Detection of the Virus by Reverse Transcription PCR (RT-PCR)

Extraction of the Total RNAs

The vegetable samples are ground in PBS-Tween®-PVP extraction buffer as described above, in a weight/volume ratio of 1/5. The ground products are centrifuged for 10 minutes at 13000 rpm. 200 μl of supernatant is transferred to another Eppendorf tube containing 20 μl of 10% sodium dodecyl sulfate (SDS), then the tubes are vortexed. After incubation for 15 minutes at 55° C., 100 μl of potassium acetate at a concentration of 3 M is added, and the tubes are placed in ice for 5 minutes. Once again, these tubes are centrifuged for 5 minutes at 13000 rpm, and the supernatant is transferred to new Eppendorf tubes. 700 μl of NaI at 6 M, then 5 μl of silica, are added. The tubes are kept at room temperature for 10 minutes, before centrifuging for 30 seconds at 5000 rpm. The pellet is washed twice with 500 μl of washing solution containing 20 mM of Tris-HCl at pH 7.5, 1 mM of EDTA, 100 mM of NaCl, and 1 equal volume of absolute ethanol. The pellets are then dried in a vacuum evaporator for 10 minutes, and then taken up in 400 μl of pure water. Finally, the tubes are incubated for 5 minutes at 55° C., and centrifuged for 5 minutes at 13000 rpm. 300 μl of supernatant is transferred to new tubes and stored at −20° C.

RT-PCR

2.5 μl of RNA extracted according to the above protocol is put in the presence of a final reaction volume of 22.5 μL containing:

0.3% (v/v) Triton 100×, 1× PCR buffer (MgCl₂ 1.5 mM, TrisHCl pH 8.4 20 mM, KCl 50 mM), 0.25 M dNTPs (Fermentas), 1 μM per primer (universal primer pair P1/P2 for PPV, P4b/P3D for PPV-D and P4b/P3m for PPV-M), 1.5 unit of RTase (Abgene) and 0.1 unit of Taq polymerase (Biolabs).

The program of the RT-PCR is as follows:

(RT)—15 minutes of reverse transcription at 42° C.
5 minutes of denaturation at 95° C.

(PCR) 40 cycles of:
denaturation at 92° C., 40 seconds
hybridization at 56° C., 40 seconds
elongation at 72° C., 40 seconds
final elongation at 72° C., 10 minutes

The primer pair P1/P2 specific to detection of PPV amplifies a 243 bp fragment of the N-terminal region of the capsid protein of the virus. The latter is detected by migration on 2% agarose gel. An RNA extracted previously and corresponding to the PPV-R isolate is used as positive control of the PCR.

Primer P1: sequence SEQ ID NO: 1

Primer P2: sequence SEQ ID NO: 2

For TuMV, the following primers CP1F and CP1R are used:

Primer CP1F: sequence SEQ ID NO: 3

Primer CP1R: sequence SEQ ID NO: 4

3-3/Observation of the Tissues Infected with the Virus in Stereomicroscopy

At the time of inoculation with the infectious clones pICPPVnkGFP and pBINPPVnkGFP fused to the GFP protein, acquisition of the fluorescence is effected under a binocular magnifying glass (Leica Microsystems, MZ FLIII, Switzerland). The filters used for visualization of the fluorescence produced are as follows:
GFP3 with 450 to 490 nm for excitation window and 500 to 550 nm for emission window.
blue with 450 to 490 nm for excitation window and emission at 515 nm.

### B/EXAMPLE 1

### Arabidopsis thaliana—csn5a Defective Mutants

The csn5a-1 and csn5a-2 mutants of Arabidopsis thaliana, described in the publication of Dohmann et al. (2005), obtained from the Columbia accession, are used.

These mutants are characterized by inactivation of the CSN5A gene (AJH1, Accession No. in GenBank: At1g22920), by insertion of T-DNA in the gene:

csn5a-1: insertion line SALK_{—}063436 in an exon of the gene;

csn5a-2: insertion line SALK_{—}027705 in an intron of the gene.

These mutants have similar phenotypes. They display reduced growth, but they are fertile and can be propagated in the form of homozygotic mutants.

The csn5a-1 mutant is characterized by the production of a truncated and inactive CSN5A protein. The csn5a-2 mutant is characterized by decreased production of the CSN5A protein.

1/Measurement of Expression of the CSN5A and CSN5B Genes by Quantitative RT-PCR

The expression of the CSN5A and CSN5B genes in each of these mutants is measured by quantitative RT-PCR, using the following primers:

For CSN5A:

Primer QAJH1 FW1: sequence SEQ ID NO: 5

Primer QAJH1 REV1: sequence SEQ ID NO: 6

For CSN5B:

Primer QAJH2FW2: sequence SEQ ID NO: 7

Primer QAJH2REV2: sequence SEQ ID NO: 8

The EF1 gene coding for the elongation factor of the translation EF1α serves as housekeeping and reference gene.

The primers used for EF1 are as follows:

Primer QAtEF1 FW: sequence SEQ ID NO: 11

Primer QAtEF1 REV: sequence SEQ ID NO: 12

Protocol of RT-PCR

a. Extraction of RNA and Synthesis of cDNA

The total RNAs were extracted from foliar tissues (100 mg) using the RNeasy® Mini Kit (QIAGEN) following the supplier's recommendations. The contaminations due to the presence of genomic DNA (gDNA) are removed by treatment with Turbo® DNAse following the supplier's protocol (Ambion). Absence of contamination by gDNA is then verified by PCR using the following specific primer pair of the ACTINE gene:

Primer Actine25: SEQ ID NO: 9

Primer Actine23: SEQ ID NO: 10

The concentration of the RNAs extracted was quantified using NanoVue® (GE Healthcare). Their quality was evaluated by electrophoresis on 1.5% agarose gel (w/v).

The cDNAs are synthesized starting from 500 ng of total RNAs in a reaction volume of 20 μl following the recommendations of the supplier of the reverse transcriptase (iScript®, Bio-Rad). The quality of the cDNAs obtained was verified as before by PCR, using the primers of the ACTINE gene.

b. Quantitative RT-PCR

Quantitative RT-PCR (Q-RT-PCR) was carried out using the GoTaq®qPCR Master Mix Kit (Promega), using the cDNAs previously obtained and diluted to 1/50th for investigating the expression of the CSN5A, CSN5B and EF1 genes. The final reaction mixture of 25 μl consists of a master mix 2× (GoTaq® Hot Start polymerase, MgCl₂, dNTP, buffer, SyBrGreen), 5 μl of cDNA and 10 μM of each primer.

Amplification is carried out in a Bio-Rad iCycler thermocycler. The samples are denatured for 3 min at 95° C. and then amplification is carried out for 40 cycles of denaturation at 95° C., for 15 seconds, and of priming/polymerization at 60° C., for 25 seconds. Finally, a step of dissociation of the amplicons from 60° C. to 95° C. is able to verify the presence of one or more species of amplicons. Production of the amplicons is followed by incorporation of SyBrGreen, the excitation of which is measured at 493 nm. The fluorescence emitted is measured at 530 nm.

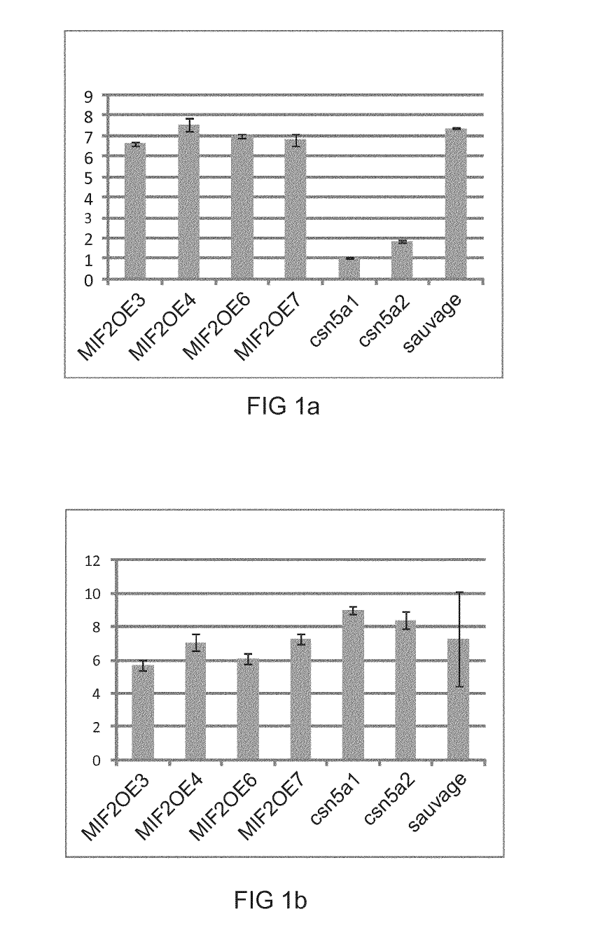

This Q-PCR is not able to quantify the number of target RNA molecules but allows the relative expression of the genes of interest to be compared between the plants investigated. The results, relative to the wild-type line, are shown in FIGS. 1a (CSN5A) and 1b (CSN5B).

It can be seen that expression of the CSN5A gene is greatly reduced relative to the wild-type line, but not inhibited completely. As for expression of the CSN5B gene, it is unaffected.

2/Tests of Resistance to Infection by the PPV and TuMV Viruses

The resistance to infection of these mutants csn5a-1 and csn5a-2 by the PPV and TuMV viruses was tested according to the protocol described above. For the mutant cns5a-1, each test was repeated three times. The positive (Columbia) and negative (E6) controls were tested simultaneously, by the same protocol. The results of the tests carried out are shown in Table 1 below.

TABLE 1
Arabidopsis thaliana - Mutants csn5a-1 and csn5a-1 - Result
of tests of infection by the PPV and TuMV viruses

| | Virus | | | |
|---|---|---|---|---|
| Line | pBINPPVnkGFP | pICPPVnkGFP | PPV-NAT | TuMV |
| csn5a-1 | 0/6-0/6-0/8 | 0/6-0/6 | 0/6 | 0/3-4/6 |
| csn5a-2 | 0/3-5/6 | 1/6 | 3/6 | 1/3 |
| Columbia | 6/6 | 6/6 | 6/6 | 6/6 |
| (positive | | | | |
| control) | | | | |
| E6 (negative | 0/6 | 0/6 | 0/6 | 0/6 |
| control) | | | | |

where x/y expresses the number x of infected plants relative to the number y of plants tested.

It is clear from the above results that both the defective mutants csn5a-1 and the defective mutants csn5a-2 have greatly improved resistance to the PPV and TuMV viruses relative to the positive control, which corresponds to the wild-type line.

### C/EXAMPLE 2

### Arabidopsis thaliana—Mutants Overexpressing MIF2

To obtain seedlings of Arabidopsis thaliana overexpressing MIF2, the Arabidopsis plants are transformed according to the following protocol.

The strain GV3101 of Agrobacterium tumefaciens is transformed, conventionally per se, by recombinant plasmids containing sense MIF2 overexpressor constructs with dependence on the 35S promoter. The pK2GW7 plasmid was used for this purpose. It is a binary vector containing the sequences of the strong constitutive promoter 35S and of the terminator of the VI gene of CaMV, between which the sequence of the MIF2 gene is inserted (GenBank accession No.: NM_{—}202644.1). This vector therefore allows overexpression of the MIF2 gene, and selection of the transformed plants is effected thanks to the selection gene conferring kanamycin resistance (Kan^{r}).

Bacteria in the middle of the exponential growth phase (OD_{600 nm}=0.6) are centrifuged at 5000 g for 10 minutes at 4° C. The supernatant is removed and the bacteria are taken up in the transformation solution (MS (Murashige and Skoog) medium pH 5.7, diluted to half, glucose 5% (w/v), Silwet L-77® 0.05% (v/v)) so as to obtain a bacterial suspension with OD_{600 nm} equal to 0.8.

The Arabidopsis seedlings are grown in earth until floral spikes of about ten centimeters are obtained. The plants thus obtained are immersed for 30 seconds in this bacterial suspension, and then kept in a humid atmosphere for 24 hours. Finally, the plants are put back in the growing room until seeds are obtained.

These seeds are collected, decontaminated and kept for 2 days at 4° C. in water containing kanamycin at 50 μg.ml⁻¹. The seeds are then taken up in lukewarm sterile agarose at 0.05% (w/v) and spread on culture medium (MS medium pH 5.7, diluted to half, agar, glucose 5% (w/v)) with addition of kanamycin at 50 μg.ml⁻¹. The kanamycin-resistant plantlets, which overexpress MIF2, are transferred into earth.

In this way, four independent lines overexpressing MIF2 were obtained (MIF2OE3, MIF2OE4, MIF2OE6, MIF2OE7).

1/Measurement of Expression of the CSN5A and CSN5B Genes by Quantitative RT-PCR

The expression of the CSN5A and CSN5B genes in these four independent lines overexpressing MIF2 (MIF2OE3, MIF2OE4, MIF2OE6, MIF2OE7) is measured by quantitative RT-PCR, according to the protocol described above in Example 2. The results, relative to the wild-type line, are shown in FIGS. 1a (CSN5A) and 1b (CSN5B).

It can be seen that, for all the mutants, expression of the CSN5A gene is little affected or unaffected relative to the wild-type line. As for expression of the CSN5B gene, it is only reduced a little, or is not reduced, relative to the wild-type line.

2/Tests of Resistance to Infection by the LMV and TuMV Viruses

The resistance of the mutants MIF2OE4 and MIF2OE7 to infection by the LMV and TuMV viruses was tested according to the protocol described above. The positive control (Columbia) and negative control (E6) were tested simultaneously, by the same protocol. The results of the tests carried out are shown in Table 2 below.

TABLE 2
Arabidopsis thaliana - Mutants overexpressing MIF2 - Result
of the tests of infection by the LMV and TuMV viruses

| | Virus | | | |
|---|---|---|---|---|
| | | | LMV | |
| Line | pBINPPVnkGFP | pICPPVnkGFP | (Var1) | TuMV-UK1 |
| MIF2OE4 | — | — | 3/10 | 2/4 |
| MIF2OE7 | — | — | 7/10 | 3/4 |
| Columbia | 6/6 | 6/6 | 8/8 | 3/4 |
| (positive | | | | |
| control) | | | | |
| E6 (negative | 0/6 | 0/6 | 0/4 | 0/4 |
| control) | | | | |

where x/y expresses the number x of plants infected relative to the number y of plants tested.

These results show that the plants overexpressing MIF2 display an improvement in resistance to infection by the LMV and TuMV viruses, relative to the positive control.

### D/EXAMPLE 3

### Tomato—Mutants Overexpressing IMA

Transformation of the tomato seedlings is carried out according to the following protocol.

The media for preculture, co-culture and regeneration used for the tomato cotyledons are described in the following Table 3.

TABLE 3
Composition of the media used for obtaining tomato mutants
overexpressing IMA

| | |
|---|---|
| Basic medium | MS medium 5 g · l⁻¹ |
| | sucrose 30 g · l⁻¹ |
| | agar 8 g · l⁻¹ |
| Preculture medium | Basic medium with addition of: |
| | IAA (indole-3-acetic acid) 0.1 mg · l⁻¹ |
| | BAP (6-benzylaminopurine) 2 mg · l⁻¹ |
| Co-culture medium | Basic medium with addition of: |
| | IAA 0.1 mg · l⁻¹ |
| | BAP 2 mg · l⁻¹ |
| Regeneration medium | Basic medium with addition of: |
| | IAA 0.1 mg · l⁻¹ |
| | BAP 2 mg · l⁻¹ |
| | TIMENTIN ® 250 mg · l⁻¹ |
| | Kanamycin 300 mg · l⁻¹ |

The tomato seeds Solanum lycopersicum are sown and grown on the MS medium (Murashige and Skoog) pH 5.7, diluted to a quarter. The cotyledons are taken from the plantlets from 7 to 9 days and cut into 3 explants.

The explants are grown for 2 days on the preculture medium, and then immersed for about thirty minutes in a culture in the exponential growth phase (OD_{600 nm}=0.6) of Agrobacterium tumefaciens transformed, conventionally per se, with one of the recombinant plasmids containing the pro35S:IMA constructs described in the publication of Sicard et al. (2008). The excess of bacterial cells is removed between two sheets of sterile absorbent paper.

The explants are then grown in the presence of the agrobacteria for 48 hours on agar co-culture medium. The explants are rinsed twice for 3 minutes in liquid MS medium, with addition of Tween®20 at 0.05% (v/v) (Sigma), then cultured on the regeneration medium until calluses are formed. The regenerated plantlets developing from the calluses are transferred onto regeneration medium lacking IAA and BAP. The kanamycin-resistant plantlets, which overexpress IMA, are transferred into earth.

The above description clearly illustrates that with its various features and the advantages thereof, the present invention provides a method for improving the resistance of plants to viruses, which is applicable to all the higher vegetable eukaryotes, and which makes it possible to obtain plants with improved resistance to all RNA viruses, in particular to the potyviruses, taking into account the cellular mechanism involved, controlled by the COP9-signalosome complex (CSN) and existing in all eukaryotic organisms.

### References

Decroocq V., Sicard O., Alamillo J-M. , Lansac M., Eyquard J-P., Garcia J-A, Candresse T., Le Gall O., Revers F. (2006) Multiple resistance traits control PPV infection in Arabidopsis thaliana. Mol. Plant Microbe Inter. 19: 541-549.
Decroocq V., B. Salvador, O. Sicard, M. Glasa, Svanella L., Cosson P., F. Revers, J. A. Garcia, T. Candresse. (2009) The determinant of potyviruses ability to overcome the RTM resistance of Arabidopsis thaliana maps to the N-terminal region of the coat protein. Molecular Plant-Microbe Interactions, 22, 1302-1311.
Dohmann E. M. N., Carola Kuhnle C., Claus Schwechheimer C. (2005) The Plant Cell, vol. 17, 1967-1978.
Duprat, A., Caranta, C., Revers, F., Menand, B., Browning, K. S. and Robaglia, C. (2002) The Arabidopsis eukaryotic initiation factor (iso)4E is dispensable for plant growth but required for susceptibility to potyviruses. Plant J. 32, 927-934.
Fernandez-Fernandez, M. R., Mouri\ddot{n}o, M., Rivera, J., Rodriguez, F., Plana-Durán, J., and Garcia, J. A. (2001) Protection of rabbits against rabbit hemorrhagic disease virus by immunization with the VP60 protein expressed in plants with a potyvirus-based vector. Virology 280:283-291.
Henikoff S, Till B. J., and Comai L. (2004) TILLING. Traditional mutagenesis meets functional genomics. Plant Physiology 135: 630-636.
Hu W. and Ma H. (2006) The Plant Journal, 45, 399-422.
Jenner, C. E., Sanchez, F., Nettleship, S. B., Foster, G. D., Ponz, F. and Walsh, J. A. (2000) The cylindrical inclusion gene of Turnip mosaic virus encodes a pathogenic determinant to the Brassica resistance gene TuRB01. Mol. Plant-Microb. Interact. 13, 1102-1108.
Jimenez, I., Lopez, L., Alamillo, J. M., Valli, A. and Garcia, J. A. (2006) Identification of a plum pox virus CI-interacting protein from chloroplast that has a negative effect in virus infection. Mol. Plant-Microb. Interact. 19, 350-358.
Krause-Sakate, R., Le Gall, O., Fakhfakh, H., Peypelut, M., Marrakchi, M., Varveri, C., Pavan, M. A., Souche, S., Lot, H., Zerbini, F. M., and Candresse, T. (2002) Molecular characterization of Lettuce mosaic virus field isolates reveals a distinct and widespread type of resistance-breaking isolate: LMV-Most. Phytopathology 92, 563-572.
Lehmann, P., Petrzik, K., Jenner, C. E., Greenland, A. J., Spak, J., Kozubek, E. and Walsh, J. A. (1997) Nucleotide and amino acid variation in the coat protein coding region of turnip mosaic virus isolates and possible involvement in the interaction with the brassica resistance geneTuRB01. Physiol. Mol. Plant Pathol. 51, 195-208.
Mac Callum et al., (2000) Plant Physiology, 123, 439-442.
Schwechheimer C., Isono E. (2010) European Journal of Cell Biology, 89, 157-162.
Sicard A., Petit J., Mouras A., Chevalier C., Hernould M. (2008) The Plant Journal, 55, 415-427.

1-13. (canceled)

14. A method for improving a plant's resistance to RNA viruses, comprising a step of modifying the plant genome so as to inhibit at least partially the activity of a CSN5 protein of said plant.

15. The method as claimed in claim 14, whereby said modification of the plant genome leads to reduction of the isopeptidase activity of said CSN5 protein.

16. The method as claimed in claim 14, wherein said CSN5 protein has at least 80% identity with peptide sequence SEQ ID NO: 13.

17. The method as claimed in claim 14, wherein said CSN5 protein has at least 80% identity with peptide sequence SEQ ID NO: 14.

18. The method as claimed in claim 14, whereby said modification of the plant genome is carried out so as to partially inhibit the activity of said CSN5 protein.

19. The method as claimed in claim 14, whereby said modification of the plant genome is a mutation of a gene of the plant encoding said CSN5 protein.

20. The method as claimed in claim 19, whereby said mutation of a gene encoding said CSN5 protein is selected for inducing a decrease in the accumulation of said CSN5 protein.

21. The method as claimed in claim 19, whereby said mutation of a gene encoding said CSN5 protein is selected for inducing the synthesis of a truncated CSN5 protein with reduced isopeptidase activity.

22. The method as claimed in claim 19, whereby said mutation of a gene encoding said CSN5 protein is introduced into an exon of said gene.

23. The method as claimed in claim 19, comprising a step of selecting, from a collection of mutant plants generated by chemical mutagenesis, viable plants possessing a mutation on said gene of the plant encoding said CSN5 protein and whose capacity for resistance to viruses is greater than that of the wild-type plant.

24. The method as claimed in claim 14, whereby said modification of the plant genome is selected for leading to overexpression of a protein that is a member of the Mini Zinc Finger protein family.

25. The method as claimed in claim 24, wherein said protein that is a member of the Mini Zinc Finger protein family has at least 60% identity with peptide sequence SEQ ID NO: 17.

26. The method as claimed in claim 24, wherein said protein that is a member of the Mini Zinc Finger protein family has at least 60% identity with peptide sequence SEQ ID NO: 18.

27. The method as claimed in claim 24, wherein said plant is the tomato, and said modification of the plant genome is selected for leading to overexpression of the protein encoded by the IMA gene of peptide sequence SEQ ID NO: 18.

28. The method as claimed in claim 14, wherein said viruses are potyviruses.


SEQUENCE LISTING (19 sequences)

SEQ ID NO: 1 - DNA, 20 bp, Artificial Sequence
  Primer
accgagaccactacactccc

SEQ ID NO: 2 - DNA, 20 bp, Artificial Sequence
  Primer
cagactacagcctcgccaga

SEQ ID NO: 3 - DNA, 20 bp, Artificial Sequence
  Primer
cgagaaaagagtggccctaa

SEQ ID NO: 4 - DNA, 21 bp, Artificial Sequence
  Primer
tcaaaccatgtgtcaaactgc

SEQ ID NO: 5 - DNA, 20 bp, Artificial Sequence
  Primer
cagatccagagcccatgatt

SEQ ID NO: 6 - DNA, 20 bp, Artificial Sequence
  Primer
taaaccgggtcaaaccgata

SEQ ID NO: 7 - DNA, 20 bp, Artificial Sequence
  Primer
caaatctcccactgactcgt

SEQ ID NO: 8 - DNA, 22 bp, Artificial Sequence
  Primer
gaaaggacaacaaatgatgggt

SEQ ID NO: 9 - DNA, 23 bp, Artificial Sequence
  Primer
aacgctgacgagtgtatggtttt

SEQ ID NO: 10 - DNA, 23 bp, Artificial Sequence
  Primer
ccaaaggtaggattagcgagctt

SEQ ID NO: 11 - DNA, 20 bp, Artificial Sequence
  Primer
gtgaagaagggtgccaaatg

SEQ ID NO: 12 - DNA, 20 bp, Artificial Sequence
  Primer
gctcaaacgccatcaaagtt

SEQ ID NO: 13 - protein, 367 aa, Solanum lycopersicum
MDSLNSYASSAAMAQQTWELENNIVTIDAPSGSKPENSASDAIFHYDDAAQTKFQREKPWASDPHYFKRVKISALALLKMVVHARSGGTIEVMGLMQGKTDGDAIIVMDAFALPVEGTETRVNAQADAYEYMVEYSQTNKQAGRLENVVGWYHSHPGYGCWLSGIDVSTQMLNQQYQEPFLAVVIDPTRTVSAGKVEIGAFRTYPEGYKPPDDPISEYQTIPLNKIEDFGVHCKQYYSLDITYFKSSLDCRLLDLLWNKYWVNTLSSSPLLGNGDYVAGQISDLAEKLEHAENQLSNSRYASLMAPPQRKKEEESQLAKITRDSAKITVEQVHGLMSQVIKDILFNSVGKSSKSQTESSGPEPMIES

SEQ ID NO: 14 - protein, 367 aa, Solanum lycopersicum
MDALNSYASSAAMAQQTWELENNIVTTDAPSGSAPENSASDAIFHYDDAAQTKFQREKPWTSDPHYFKRVKISALALLKMVVHARSGGTIEVMGLMQGKTDGDAIIVMDAFALPVEGTETRVNAQADAYEYMVEYSQTNKQAGRLENVVGWYHSHPGYGCWLSGIDVTTQMLNQQYQEPFLAVVIDPTRTVSAGKVEIGAFRTYPEGYKPPDDPISEYQTIPLNKIEDFGVHCKQYYSLDITYFKSSLDCHLLDLLWNKYWVNTLSSSPLLGNGDYVAGQISDLAEKLEQAENQLSHSRFGHLVAAPQRKKEEESQLAKITRDSAKITVEQVHGLMSQVIKDILFNSVCKSGKSQTEPSDPEPMVET

SEQ ID NO: 15 - protein, 357 aa, Arabidopsis thaliana
MEGSSSAIARKTWELENNILPVEPTDSASDSIFHYDDASQAKIQQEKPWASDPNYFKRVHISALALLKMVVHARSGGTIEIMGLMQGKTEGDTIIVMDAFALPVEGTETRVNAQSDAYEYMVEYSQTSKLAGRLENVVGWYHSHPGYGCWLSGIDVSTQMLNQQYQEPFLAVVIDPTRTVSAGKVEIGAFRTYPEGHKISDDHVSEYQTIPLNKIEDFGVHCKQYYSLDITYFKSSLDSHLLDLLWNKYWVNTLSSSPLLGNGDYVAGQISDLAEKLEQAESQLANSRYGGIAPAGHQRRKEDEPQLAKITRDSAKITVEQVHGLMSQVIKDILFNSARQSKKSADDSSDPEPMITS

SEQ ID NO: 16 - protein, 358 aa, Arabidopsis thaliana
MEGSSSTIARKTWELENSILTVDSPDSTSDNIFYYDDTSQTRFQQEKPWENDPHYFKRVKISALALLKMVVHARSGGTIEIMGLMQGKTDGDTIIVMDAFALPVEGTETRVNAQDDAYEYMVEYSQTNKLAGRLENVVGWYHSHPGYGCWLSGIDVSTQRLNQQHQEPFLAVVIDPTRTVSAGKVEIGAFRTYSKGYKPPDEPVSEYQTIPLNKIEDFGVHCKQYYSLDVTYFKSSLDSHLLDLLWNKYWVNTLSSSPLLGNGDYVAGQISDLAEKLEQAESHLVQSRFGGVVPSSLHKKKEDESQLTKITRDSAKITVEQVHGLMSQVIKDELFNSMRQSNNKSPTDSSDPDPMITY

SEQ ID NO: 17 - protein, 100 aa, Arabidopsis thaliana
MRKRQVVLRRASPEEPSRSSSTASSLTVRTVRYGECQKNHAAAVGGYAVDGCREFMASRGEEGTVAALTCAACGCHRSFHRREIETEVVCDCNSPPSTGN

SEQ ID NO: 18 - protein, 90 aa, Solanum lycopersicum
MKKVLRRNDYSRNSTNSSFTMRRVRYVECQRNHAASVGGYVIDGCREYMPEGTTSGTLNCAACGCHRNFHRREVETDVASECTSASSTTK

SEQ ID NO: 19 - protein, 366 aa, Prunus persica
MDAQIAQQTWELENNIIPMDTPPSVAKSKADPSADAIFYYDEAAQAKFQQEKPWTNDPHYFKRVKISALALLKMVVHARSGGTIEVMGLMQGKTDGDAIIVMDAFALPVEGTETRVNAQADAYEYMVDYSQTNKQAGRLENVVGWYHSHPGYGCWLSGIDVSTQMLNQQFQEPFLAVVIDPTRTVSAGKVDIGAFRTYPEGYKPPDDPVSEYQTIPLNKIEDFGVHCKQYYSLDITYFKSSLDCHLLDLLWNKYWVNTLSSSPLLGNGDYVAGQISDLAEKLEQAENHLSHSRFGPLITPPQRKKEEESQLAKITRDSAKITVEQVHGLMSQVIKDILFNSVRQSNKSLTQPSLTEPSDPEPMVES